(19) United States
(12) Patent Application Publication
(10) Pub. No.: US 2004/0161422 A1
(43) Pub. Date: Aug. 19, 2004

(54) Nutritional compositions comprising probiotics

(76) Inventor: Natarajan Ranganathan, Broomall, PA (US)

Correspondence Address: Jane Massey Licata, Licata & Tyrrell P.C., 66 E. Main Street, Marlton, NJ, 08053, US

(21) Appl. No.: 10/689,359

(22) Filed: Oct. 20, 2003

Related U.S. Application Data
(63) Continuation-in-part of application No. 09/855,346, filed on May 15, 2001, now Pat. No. 6,706,287.
Application No. 09/855,346 is a continuation-in-part of application No. 09/557,011, filed on Apr. 20, 2000, now Pat. No. 6,706,263.
(60) Provisional application No. 60/131,774, filed on Apr. 30, 1999.

(51) Int. Cl. A61K 35/74

(52) U.S. Cl. 424/93.45

(57) ABSTRACT

The present invention provides a food or nutritional product for consumption by individuals who want to maintain a healthy gastrointestinal tract, the probiotic composition is efficacious in removing toxic nitrogenous by-products of metabolism. Embodiments of the invention further include health bars yogurt, yogurt based products and foods that contain one or more vitamins or minerals, in addition to a carbohydrate, fats and protein components.

### CROSS REFERENCE TO RELATED APPLICATIONS

This application is a continuation-in-part of U.S. patent application Ser. No. 09/855,346 filed May 15, 2001 which is a continuation-in-part of U.S. patent application Ser. No. 09/557,011 filed Apr. 20, 2000, which claims the benefit of priority of U.S. Provisional Application 60/131,774 filed Apr. 30, 1999.

### GOVERNMENT SUPPORT

This work has been partly funded from the Small Business Innovation Research (SBIR) grant from the National Institutes of Diabetes, Digestive and Kidney (NIDDK) Diseases, National Institute of Health (NIH), Bethesda, Md., Grant No: 1R44DK61873-01.

### FIELD OF THE INVENTION

The present invention relates to nutritional or medical food products which provide probiotics to the gastrointestinal tract of a subject. More particularly, the present invention relates to a nutritional health bar for providing probiotics to metabolize nitrogenous waste products, such as urea and ammonia, to promote and/or enhance gastrointestinal health.

### BACKGROUND OF THE INVENTION

The human gastrointestinal tract harbors a complex microbial ecosystem containing a large number and variety of bacteria. The resident bacterial population in the human gastrointestinal tract has a major impact on gastrointestinal function and thereby on human health and well being. Among these, some bacteria are opportunistic or considered to be detrimental and cause adverse conditions such as diarrhea, infections, gastroenteritis and endotoxaemia, while other bacteria are considered “probiotic”, in that they perform beneficial functions for the human organism (Holzapfel W H, et al. Int J Food Microbiol 1998 May 26; 41(2): 85-101).

Probiotic bacteria are known to stimulate the immune system and exert a competitive exclusion of pathogenic and putrefactive bacteria, reduce the amounts of ammonia and cholesterol in the blood, and promote absorption of minerals (von Wright, et al. Eur J Gastroenterol Hepatol 1999 Nov; 11(11): 1195-1198). Additionally, probiotic bacteria produce antagonist effects against pathogenic microorganisms; stimulate the immune system; improve lactose digestion; are lypolytic, thereby allowing fats to be more digestible; reduce plasma cholesterol; protect the intestinal mucosa, thereby assuring effective assimilation of the nutritive substances; produce polysaccharides that are active on some tumors; and reduce viability of some enzyme-producing microorganisms which catalyze the conversion of procarcinogenic substances into carcinogenic substances.

It is believed that the probiotic bacteria exert their effects in a synergistic manner to curtail and retard the growth of pathogenic and detrimental bacteria of the gut (Marteau, PR et al. Am J Clin Nutr Feb; 73(2 Suppl): 430S-436S; Cummings J H, et al. Am J Clin Nutr 2001 Feb; 73(2 Suppl): 415S-420S).

It is the contention of many scientists that the health and well being of people can be positively or negatively influenced by the microorganisms which inhabit the gastrointestinal tract, and in particular the large bowel. These microorganisms through the production of toxins, metabolic by-products, short chain fatty acids, and the like affect the physiological condition of the host. The constitution and quantity of the gut microflora can be influenced by conditions such as, but not limited to, diabetes and renal insufficiencies. If microorganisms which positively affect the health and well being of the individual can be encouraged to populate the large bowel, this will improve the physiological well being of the host.

The present invention provides a functional supplement in the form of a nutritional food or nutritional product with a long shelf life at non-refrigerated temperatures which contains probiotics. In one embodiment of the invention the nutritional food or nutritional product contains probiotics along with prebiotics. The nutritional supplement provided herein allows for a greater population of the probiotic bacteria to colonize the gastrointestinal tract. Thus, the present invention supplies a long sought, yet unfulfilled need to the public for a convenient source of beneficial bacteria.

### DETAILED DESCRIPTION OF THE INVENTION

The nutritional compositions and methods of the present invention are directed to the use of probiotics in tasty, ready-to-eat food or nutritional products. The nutritional compositions of the present invention comprise probiotics that, when ingested, effectively enhance or improve gastrointestinal health. The term “food or nutritional composition” refers to any probiotic containing consumable food, such as, but not limited to, food bars, sticks, pastes, cookies, cakes, pies, breads, cupcakes, muffins, biscuits, chips, shakes, candies, prepared snack foods, and the like, either baked or not, intended to be eaten by a human or other animal.

“Probiotic bacteria” as used herein are living microorganisms that are naturally present in the food chain like yogurt and other fermented foods, and in the gastrointestinal tract of humans and animals. They are beneficial bacteria that enhance the body's defenses against a number of health conditions.

“Prebiotic” as used herein is meant to mean any nonliving, non-digestible food ingredient that beneficially affects the host by selectively stimulating the growth and/or activity of one or a limited number of bacteria, including probiotic bacteria, in the colon, with the effect of improving the host's health.

“Effective amount” as used herein is meant to mean an amount necessary to achieve a selected result. For example, an effective amount of a bacteria and fiber-containing composition useful for reducing pathogenic microorganisms in the gastrointestinal tract is an amount that achieves the selected result of reducing the pathogenic microorganisms. Such an amount is readily determined without undue experimentation by those skilled in art.

The present invention provides a food or nutritional product comprising at least one probiotic bacteria, at least one carbohydrate ingredient, at least one fat ingredient, and at least one protein ingredient. In another embodiment the food or nutritional product of the present invention which effectively maintains or enhances gastrointestinal health, comprises at least one carbohydrate ingredient, at least one fat ingredient, at least one protein ingredient, at least one vitamin component, at least one mineral component, at least one prebiotic ingredient, and at least one probiotic bacteria, wherein said probiotic bacteria has a propensity to hydrolyze nitrogenous waste products.

In one aspect of the present invention the food or nutritional product is in the form of a food bar which is easily consumed, portable and convenient. The food or nutritional product of the present invention may be in the form of a dietary supplement, a nutriceutical product, a medical food or a functional food. Food bars may be formed by compression, extrusion, or other methods that are well known to those of skill in the art. Other forms of food, yogurt or nutritional products are also well known to those of ordinary skill in the art, and the methods of the present invention may easily be adapted to produce food or nutritional products in forms other than food bars.

The carbohydrate ingredient may comprise dextrose, glucose, sucrose, fructose, lactose, maltose, galactose, sugar alcohols, such as sorbitol, mannitol,xylitol, invert sugar syrups, brown sugar, corn syrup, corn syrup solids, honey, molasses, brown sugar, maple syrup, fruit juices, stevia, or an artificial sweetener.

The fat ingredient may comprise olive oil, canola oil, palm oil, coconut oil, sunflower oil, peanut oil, vegetable oil, lecithin, fish oil, cotton seed oil, soybean oil, lard, monoglycerides, diglycerides, butter, margarine, other animal, vegetable, marine fats, or milk fats.

The protein ingredient may comprise cereal proteins, milk proteins, egg proteins, animal proteins, vegetable proteins, whey protein, bean proteins, lactalbumin-casein coprecipitate, calcium caseinate, sodium caseinate, purified or refined grades of casein, soy proteins, or peanut.

The prebiotic ingredient may comprise a fructo-oligosaccharide, a galacto-oligosaccharide, a soy-oligosaccharide, a xylo-oligosaccharide, a isomalto-oligosaccharides, Jerusalem artichoke flour, rolled oats, banana fiber, a pectin, pectic polysaccharide, a mannan, a pentosan, a beta-glucan, a rabinan or a galactan. The present invention may be further fortified with vitamin components or mineral components.

In one embodiment of the present invention the health food product is formulated with bacterial strains that have a greater propensity to hydrolyze nitrogenous waste products than the probiotic bacteria present within currently available supplements. Organisms with a greater propensity to hydrolyze nitrogenous waste products include, but are not limited, to a selected strain of Lactobacillus acidophilus, Lactobacillus rhamnosus, Bifidobacterium, Streptococcus thermophilus, Bacillus coagulants, Lactobacillus caseii, or Lactobacillus ruteri.

In a further embodiment of the invention, the food or nutritional product further comprises one or more prebiotic components which are included with the probiotic supplement. A prebiotic component is any soluble dietary fiber or insoluble fiber that has a favorable influence on the growth of certain bacteria.

The probiotic bacteria may comprise at least one of Lactobacillus acidophilus, L. bulgaricus, L. casei, L. rhamnosus, L. fermentum, L. salivaroes, L. brevis, L. plantarum, L. ruteri, S. thermophilus, Bacillus sporogenes, Bifidobacterium adolescentis, B. infantis, B. longum, B. thermophilum, or B. bifidum. The nutritional food or nutritional product provides about 5 billion to 20 billion colony forming units of said at least one probiotic bacteria. Bifidobacterium is one example of a probiotic bacteria which inhibits colonization of invading pathogens in the intestine. Bifidobacterium fiercely compete for nutrients and attachment sites on the intestinal tract surface. Additionally, these bacteria produce natural antibiotics to fight off any pathogenic organism. Bifidobacterium has the added benefit of assisting in the absorption of calcium, vitamins and minerals which help rid the body of toxins. Lactobacillus species of microorganisms possess the ability to produce lactic acid. This is key to the effectiveness of the probiotic lactic acid-producing bacteria. Lactic acid production markedly decreases the pH (i.e., increases acidity) within the local micro-floral environment and does not contribute to the growth of any undesirable, physiologically-deleterious bacteria or fungi. Thus, by the mechanism of lactic acid production, the probiotic lactic acid bacteria inhibit the growth of competing pathogenic bacteria. Exemplary lactic acid-producing Lactobacillus species include, but are not limited to: Lactobacillus acidophilus, Lactobacillus casei, Lactobacillus rhamnosus, Lactobacillus plantarum, Lactobacillus reuteri, Lactobacillus gasserii, Lactobacillus jensenii, Lactobacillus caseii, Lactobacillus bulgaricus, Lactobacillus salivarius or Lactobacillus sporogenes (also designated as Bacillus Coagulants).

Lactobacillus reuteri is a dominant heterofermentative Lactobacillus species residing in the gastrointestinal tract of healthy humans and most animals. Like other lactobacilli, L. reuteri produces acidic metabolic end-products which have considerable antimicrobial activity. The metabolism of glycerol by L. reuteri can result in excretion of a metabolic intermediate, 3-hydroxpropionaldehyde, or reuterin. Reuterin has been shown to have antimicrobial activity against a variety of organisms including gram-positive or gram-negative bacteria, yeast, molds or protozoa. It is believed that the antimicrobial activity of reuterin contributes to the survival of L. reuteri within the gastrointestinal ecosystem.

Lactobacillus acidophilus is one of the prominent microorganisms in the small intestines. In a preferred embodiment, the present invention is a food or nutritional product comprising L. acidophilus. L. acidophilus has a strong ability to adhere to the intestinal wall of a subject, thereby allowing for rapid colonization. These microorganisms also produce anti-microbial substances (natural antibiotics). The beneficial effect of L. acidophilus is further illustrated by data showing that L. acidophilus inhibits the toxic activities of bacteria in patients with chronic kidney failure. Such patients often have toxic levels of amines in their blood due to bacterial overgrowth in the small bowel. Consumption of high levels of freeze dried bacteria drastically reduced levels of these toxic amines. These results demonstrate the ability of L. acidophilus to exert a positive effect on the microflora of the intestines.

Streptococcus thermophilus is a major component of cheeses. It is also a probiotic bacteria that is used to make yogurt. The strain of S. thermophilus used in the formulation of the present invention is able to survive and utilize or degrade urea at pH ranges of between about 5 and 6.5, typically found in the small intestine. At these pH levels the organism retains the ability to utilize urea as a nitrogen source, and thus benefit those individuals that have or are prone to the accumulation of toxic nitrogenous waste products.

Toxic nitrogenous waste products can accumulate in the intestinal tract of a subject when the normal balance of intestinal microbes is disrupted. Nitrogenous waste products include, but are not limited to, urea, uric acid, creatinine, ammonia, etc. These products will also tend to accumulate in the gastrointestinal tract in any condition which disrupts the kidneys ability to excrete the build up of nitrogenous waste products in the blood, thereby resulting in the diffusion of the nitrogenous waste products from the circulating blood into the bowel. Exemplary conditions that affect nitrogen metabolism include, but are not limited to, defects in protein metabolism, nucleic acid metabolism, etc. Diabetes, kidney failure, and liver disease, as well as other conditions can result in the build up of toxic nitrogenous compounds in the blood. Thus, the present invention provides a convenient, ready to eat supplement that will supply the necessary probiotics to aid in the removal of these toxic nitrogenous byproducts. The probiotics in the health bars or health foods of the present invention have a greater propensity than probiotics currently incorporated into supplements, to allow for the efficient hydrolysis of toxic nitrogenous products so that urea, uric acid, creatinine, etc. (examples of toxic nitrogenous products) are safely and effectively removed from the fecal matter of a subject. Thus, the present invention provides a long sought, yet unfulfilled need for a probiotic supplement wherein the supplement is in the form of a tasty, nutritious health bar or health food.

Moreover, the probiotic formulation in the health bar or health food provided herein will benefit the overall general condition of the gastrointestinal tract. This is especially beneficial to individuals with a propensity to gastrointestinal complaints. Exemplary gastrointestinal complaints include, but are not limited to, inflammatory conditions, such as, but not limited to, irritable bowel syndrome, inflammatory bowel disease, colitis, Crohn's disease; and diarrhea.

In one aspect the present invention comprises a nutriceutical composition which alleviates the symptoms of uremia in a subject. The nutriceutical composition comprises a composition of a probiotic, a prebiotic, and an ammoniaphilic urea degrading microorganism with pH stability and urea degrading activity. The nutriceutical composition may be in the form of a gel cap, capsule, pill or other suitable form stable to the gastrointestinal conditions and so that the nutriceutical composition is delivered into the large intestines of a patient. The nutriceutical composition may be microencapsulated or enteric coated with a material designed to deliver the probiotic, prebiotic, and ammoniaphilic urea degrading microorganism to their site of action in relatively native form without binding of digestive materials to the composition prior to reaching the target region, wherein said prebiotic ensures the viability of the probiotic, and wherein said microencapsulated or enteric prevents the release of the ammoniaphilic urea degrading microorganism into the patient.

Finally, the present invention is also meant for individuals with no gastrointestinal complaints, as an aid to maintain a healthy gastrointestinal tract.

A healthy gastrointestinal tract with adequate mucus production and appropriate bacterial colonization prevents the overgrowth of pathogenic bacteria, modulates disease processes, and prevents widespread inflammatory disorders. A complex bacterial structure and diverse pecking order exist among the microorganisms that inhabit the gastrointestinal tract. To maintain and promote a healthy gastrointestinal tract it is important to effectively encourage the growth of beneficial bacteria while minimizing the proliferation and/or overgrowth of the detrimental bacteria.

The probiotic composition of the food or nutritional product of the present invention will limit the growth of the detrimental bacteria while supplying the necessary beneficial bacteria. Additionally, the probiotics presented in the food or nutritional product provided herein have an increased ability to hydrolyze nitrogenous waste products.

Thus, not only will a healthy population of individuals benefit from ingestion of the health bar provided herein, but those individuals who may be prone or predisposed to develop kidney failure, whether from diabetes or some other condition, will have the added benefit of inhibiting the development of toxic uremia. For example, the probiotic bacteria of the present invention will remove excess nitrogenous waste products of protein metabolism, thereby reducing the burden on an ailing kidney. Additionally, ammonia is also removed, thereby avoiding potential health problems such as mental retardation and related conditions.

Foods and nutritional compositions of the present invention are supplied in various forms including a health bar, health shake, yogurt or yogurt-based preparation or other similar forms of health foods. In one embodiment, the yogurt or yogurt based product comprises at least one probiotic bacteria, at least one carbohydrate ingredient and at least one protein. The yogurt or yogurt-based product may vary in protein or fat content. The yogurt or yogurt based product may be non-fat. The yogurt or yogurt based product may further comprise fruits, bran, granola or flavorings. The consistency of the yogurt or yogurt based product may vary depending upon the type of nutritional compositions required.

The foods or nutritional products of the present invention provide a ready-to-eat, portable supplements containing probiotic bacteria for the maintenance and restoration of gastrointestinal health. The probiotic formulation provided by the present invention is selected so that the bacteria have an increased capability to hydrolyze nitrogenous compounds, such as those that accumulate during certain diseased processes. Thus, the bacterial formulation presented herein will aid in decreasing any build-up of toxins and metabolic wastes that may have accumulated. Additionally, the probiotics contained in the food or nutritional products of the present invention will inhibit, and thus decrease, any potential for the overgrowth of undesirable bacteria.

As disclosed herein, probiotics may be present in the food or nutritional products with other ingredients as necessary to increase the shelf life of these ready-to-eat foods. The term “shelf-life,” as used herein, refers to the length of time after a food or nutritional product is manufactured and/or packaged until it becomes unsuitable for sale, due to staleness, microbial spoilage, oxidation, separation of ingredients, or other causes. For example, light, heat, oxygen and moisture content will adversely affect the viability of the probiotic bacteria.

Incorporation of the bacteria into a food or nutritional product will protect them from these adverse conditions similar to pill or powder formulations.

Additionally, consumption of probiotics with food, such as within the food or nutritional product provided herein, will allow these bacteria to colonize the intestinal tract at a much lower dosage than when they are in a pill or powder supplement. Thus, an effective dosage of active, viable bacteria is provided by the food or nutritional product provided by the present invention.

Human nutritional bars have been introduced which provide the minerals and vitamins recommended by the U.S. Government for good health. However, these bars have generally been high in moisture, resulting in a limited shelf life. Furthermore, these bars do not contain probiotic supplements. One health bar sold only in Canada called ‘Dr. Gelda's Bifidus Energy Bar’ (Gelda Scientific and Food, Ontario, Canada) does contain probiotic bacteria. This bar contains 250 million colony forming units of B. longum and B. infantis and is said to provide “a step towards better health”. The Dr. Gelda's Bifidus Energy Bar only provides a small number (250 million CFU's) of probiotic bacteria “for better health”. In contrast, the invention provided herein provides a food or nutritional product wherein a high number of probiotic bacteria about 5 to 20 billion CFU's provide not only good gastrointestinal health and overall well-being, but they have a greater propensity to hydrolyze toxic nitrogenous waste products.

The special properties of the probiotic bacteria provided by the present invention such as, the enhanced ability to hydrolyze nitrogenous waste products, have not been found in any other food or supplement. The ability to hydrolyze nitrogenous waste products is especially beneficial to those individuals prone to develop, or which have developed, a condition that has resulted in the build up of toxic nitrogenous compounds. Thus, the present invention meets a long sought need to restore and maintain gastrointestinal health by providing a tasty, ready to eat food or nutritional product with the necessary probiotic bacteria. The food or nutritional product provided herein has the added benefit of aiding in the removal of nitrogenous waste products, which is especially beneficial to those individuals having or prone to have the buildup of toxic nitrogenous waste products in the circulating blood.

Daily intake of probiotic supplements maintains and restores gastrointestinal health. The current state of the art provides probiotics in the form of a pill, capsule, tablet or in a powdered form. The probiotic bacteria in these types of supplements is prone to loss in viability due to their greater susceptibility to moisture, light, oxygen, and heat. Additionally, in the absence of food a greater number of bacteria are required to colonize the gastrointestinal tract. The invention provided herein overcomes these problems by providing a solid, low-moisture nutritional food or nutritional product. Additionally, since the probiotics are consumed within a food the number of bacteria required for colonization is reduced.

A method of restoring and maintaining gastrointestinal health comprising eating at least one food or nutritional product comprising an effective amount of probiotic bacteria and an effective amount of a prebiotic is provided by the present invention.

In one embodiment of the present invention the food or nutritional product is further formulated to provide 100% of the recommended daily allowances of one or more vitamins or minerals (including micro-nutrients).

The food or nutritional product of the invention comprises the probiotic formulation in a base that includes, but is not limited to, a sugar-based sweetening ingredient or an artificial sweetening ingredient, a quantity of fat, or other desired nutrients such as proteins, vitamins, minerals, or additives. An additional embodiment further includes at least one prebiotic component.

A wide variety of carbohydrates can be employed to produce the health bars/foods of the invention. These carbohydrates include any carbohydrates that are customary in the preparation of foods, such as, but not limited to: ingestible monosaccharide or disaccharide materials, their hydrolysis products, and mixtures thereof, for example, dextrose (glucose), sucrose, fructose, lactose, maltose, galactose, sugar alcohols, such as sorbitol, mannitol, xylitol, invert sugar syrups, brown sugar, corn syrup, corn syrup solids, honey, molasses, brown sugar, maple syrup, fruit juices, stevia or commercially available carbohydrates from sources known by those of skill in the art, and mixtures thereof. The carbohydrate ingredients can also be provided with flavorings (such as apple or licorice flavored bars for horses and humans) as may be desired for the particular application. Alternatively, an artificial sweetening ingredient may be added for a low or lower calorie food or nutritional product . Artificial sweeteners include, but are not limited to, aspartame, salts of acesulfame, altitame, saccharin and its salts, cyclamic acid and its salts, glycerrhizinate, dihydrochalcones, thaumatin, monellin, and the like, alone or in combination. The range of these sweeteners in the food or nutritional product formulations range from about 0.02% to about 0.10% for alitame, thaumatin and dihydrochalcones, and from about 0.1% to about 0.2% for aspartame, sucralose, acesulfame and saccharin. These weights are based upon the total weight of the bar taken as 100% by weight. Combinations of sugar and/or sugarless sweeteners may also be used. The embodiments that contain low calorie sweeteners will also contain a low caloric bulking agent. Examples of low caloric bulking agents include, but are not limited to: polydextrose; Raftilose, Raftilin; Fructooligosaccharides; Palatinose oligosaccharide; Guar Gum Hydrolysate; or indigestible dextrin.

One embodiment of the food or nutritional product of the invention is in the form of bars which include, in one embodiment from about 47-82% by weight of a sugar-based carbohydrate ingredient. In another embodiment the food or nutritional product contains about 60-78% by weight of a sugar-based carbohydrate ingredient. In yet another embodiment there is from about 65-73% by weight of a sugar-based carbohydrate ingredient. These weights are based upon the total weight of the bar taken as 100% by weight.

The health bars/foods include a total fat (as used herein, “fat” is intended to include both fats and oils) content of from about 2.0-12.0% by weight. In another embodiment the fat content is from about 3.0-5.0% by weight. These weights are based upon the total weight of the bar taken as 100% by weight. At least a portion of this fat is an edible, hydrogenated vegetable oil or a product derived from such a vegetable oil. In one embodiment the hydrogenated vegetable oil is present at a level of from about 0.5-1.0% by weight. In another embodiment the edible fat is from about 0.7-0.8% by weight. These weights are based upon the total weight of the bar taken as 100% by weight. The hydrogenated vegetable oil acts as a moisture barrier and lubricant for the bars. These edible oils have the added benefit of protecting the probiotic bacteria from moisture, which is detrimental to maintaining a viable population of bacteria.

A wide variety of edible proteins may be employed to produce the products of the present invention. These proteins include, for example, but not limited to, cereal proteins, milk proteins, egg proteins, animal proteins, vegetable proteins, whey protein, bean proteins, lactalbumin-casein coprecipitate, calcium caseinate, sodium caseinate, purified or refined grades of casein, soy proteins, or peanuts, which are commercially available from sources known by those of skill in the art, and mixtures thereof.

The proteins employed to produce the health bars/foods of the invention range from about 5% to about 80% of the total weight of the health bars/foods. In another embodiment the protein ingredient(s) range from about 20% to about 60%. In a further embodiment the protein component(s) are about 40%. Low-, no- or high-protein health bars/foods are also provided by the invention herein.

The vitamins and minerals (“minerals” as used herein includes macro- and micro-nutrients) are present in one embodiment of the food or nutritional product of the invention at a level of from about 0.5-2% by weight. Another embodiment contains from about 0.5-1.0% by weight. All weights are based upon the total weight of the bar taken as 100% by weight. Any vitamins or minerals are added to the bar as desired, including, but not limited to: magnesium, selenium, calcium, copper, and both fat soluble (vitamin A, D, E, etc.) and water soluble vitamins (vitamin C, the B vitamins, etc.). Those skilled in the art will appreciate that, in addition to vitamins and minerals, another embodiment of the bars of the invention will supply amino acids or large quantities of protein.

Suitable prebiotics include, but are not limited to, oligosaccharides such as fructo-oligosaccharides (such as, but not limited to, inulin), galacto-oligosaccharides, soy-oligosaccharides, xylo-oligosaccharides, isomalto-oligosaccharides, Jerusalem artichoke flour, rolled oats, banana fiber, pectins, pectic polysaccharides, mannans, guar gum, locust bean gum, konjac, and xanthan gum, pentosans, beta-glucans, arabinans or galactans, larch arabinogalactans, and mixtures thereof. In one embodiment the food or nutritional product of the present invention contains from about 2.0%-6.0% prebiotic component(s). In another embodiment the prebiotics will be from about 3.0%-5.0%. An additional embodiment will contain about 4.0%-5.0%. All weights are based upon the total weight of the bar taken as 100% by weight.

The beneficial intestinal microorganisms include at least one of a selected bacterial strain, such as Lactobacillus acidophilus, L. bulgaricus, L. casei, L. rhamnosus, L. fermentum, L. salivaroes, L. brevis, L. plantarum, L. ruteri, S. thermophilus, Bacillus sporogenes, Bifidobacterium adolescentis, B. infantis, B. longum, B. thermophilum, or B. bifidum.

The food or nutritional product of the present invention contains about 5 billion to 20 billion colony forming units (CFU) of viable bacteria. In another embodiment the food or nutritional product will contain about 5 billion to 9 billion CFU. In a further embodiment the food or nutritional product will contain about 6 billion to 8 billion CFU of viable bacteria.

The bars of the invention are unique in that they are formulated into probiotic containing bars. In one embodiment these bars are scored into bite-size squares which can easily be broken apart and consumed. The bars of the invention have overall dimensions in the range of about 6 inches long, about 1.5 inches wide, and 0.5 inches thick.

In order to ensure a long shelf life, one embodiment of the bars should have a total moisture content of less than about 5% by weight. In another embodiment the moisture content is from about 2-4% by weight. These weights are based upon the total weight of the bar taken as 100% by weight. In one embodiment of the invention the water activity of the bars of the invention is less than about 0.47. The water content in another embodiment is less than about 0.43.

The food or nutritional products of the invention are made by forming a mixture comprising a sweetening ingredient and fat, followed by cooking the resulting liquid mixture. The cooked mixture is then cooled prior to mixing with the viable probiotic bacteria in order to minimize or prevent loss of viability.

The dry ingredients are added to the cooled mixture either along with or following the addition of the viable probiotic bacteria. In one embodiment of the invention the dry ingredients are from about 10-47% by weight, preferably 25-38% by weight, or from about 30-35%. These percentages are based upon the total weight of all ingredients used taken as 100% by weight.

The food or nutritional products of the present invention can be ingested by a subject at least once during a twenty four hour period, or as need to enhance health. In another embodiment the food or nutritional product of the present invention is ingested by a subject at least twice during a twenty four hour period. In a further embodiment the food or nutritional product is ingested by a subject at least three times during a twenty four hour period. The intake of the food or nutritional product provided herein is not restricted to 1, 2, or 3 times in a twenty four hour period. The precise amount of the food or nutritional products of the present invention ingested by a subject may be decided according to the judgment of a practitioner or dietician and each patient's circumstances.

### EXAMPLES

### Example 1

Kibow Bar Recipe for Preparation under Room and Lower Storage Temperature

1 C Oat Bran

½ C Toasted Sunflower and/or Sesame seeds, or peanut suitably ground in a dry grinder or kitchen mill

½ C Low fat milk or Soy Milk Powder—may be added with maltodextrin or fructose or aspartame to appropriate sweetness

½ C Raisins plus 2 Tablespoon Carob Powder

Mix well, then add to ½ C white or Brown Rice, Cooked, dehydrated and pulverized and (using a food processor again) ½ C Peanut Butter (more or less, depending on consistency) ½ C Honey or high fructose corn syrup, about 1 ml of Vanilla essence (natural). Stir all ingredients and knead (if needed add more Oat Bran or Rolled Oats) until thoroughly mixed. A cake mixer works well for this. To this add (sprinkle or spray) the microbial cocktail powder mix (approximately 10 billion cfu/gm). Mix well again. The bars can be reasonably soft, and cooling in the fridge for about 24 hrs will help to bind all ingredients together. Roll or press out about 1 cm thick and cut into suitable size bars. Makes about 16, with the preferred size of approx 1 cm×1.5 cm×6 cm.

### Example 2

Kibow Bar Recipe for Non-refrigerated Conditions

Preparation at 35° C. to 40° C. for a brief period of time and storage preferably at room temperature (non-refrigerated conditions). Combine all the dry ingredients, mix uniformly. To this add all the liquid ingredients and heat to approximately 50° C. to 60° C. Allow the mixture to cool with continued mixing. The product will become more and viscous. When the temperature is between 35° C. to 40° C., add the microbial cocktail mixture (approximately 10 billion cfu/gm). Pour the entire product in a tray lined with a cookie sheet or lightly greased with clarified butter. Immediately allow it to cool and settle in the fridge for 12 to 24 hours until sufficiently hard but easy to cut into required size. Cut to desired pieces and dimensions. Wrap and pack individually.

Although the foregoing invention has been described in some detail by way of illustration and example for the purposes of clarity of understanding, it will be readily apparent to those of ordinary skill in the art in light of the teachings of this invention that certain minor changes and modifications may be made thereto without departing from the spirit or scope of the appended claims. In addition, large scale or commercial scale up of this invention could easily be accomplished by those of ordinary and specialized skills possessing the needed equipment and facilities.

What is claimed is:

1. A nutritional food or nutritional product comprising at least one probiotic bacteria, at least one carbohydrate ingredient, at least one fat ingredient, and at least one protein ingredient.

2. The nutritional food or nutritional product of claim 1 wherein said at least one probiotic bacteria is Lactobacillus acidophilus, L. bulgaricus, L. casei, L. rhamnosus, L. fermentum, L. salivaroes, L. brevis, L. plantarum, L. ruteri, S. thermophilus, Bacillus sporogenes, Bifidobacterium adolescentis, B. infantis, B. longum, B. thermophilum or B. bifidum and wherein said nutritional food or nutritional product provides about 5 billion to 20 billion colony forming units of said at least one probiotic bacteria.

3. The nutritional food or nutritional product of claim 1 wherein said at least one carbohydrate ingredient is dextrose, sucrose, fructose, lactose, maltose, galactose, sugar alcohols, such as sorbitol, mannitol and xylitol, invert sugar syrups, brown sugar, corn syrup, corn syrup solids, honey, molasses, brown sugar, maple syrup, fruit juices, stevia, or an artificial sweetener.

4. The nutritional food or nutritional product of claim 1 wherein said at least one fat ingredient is olive oil, canola oil, palm oil, coconut oil, sunflower oil, peanut oil, vegetable oil, lecithin, fish oil, cotton seed oil, soybean oil, lard, monoglycerides, diglycerides, butter, margarine, and other animal, vegetable, and marine fats, or milk fats.

5. The nutritional food or nutritional product of claim 1 wherein at least one protein ingredient is cereal proteins, milk proteins, egg proteins, animal proteins, vegetable proteins, whey protein, bean proteins, lactalbumin-casein coprecipitate, calcium caseinate, sodium caseinate, purified or refined grades of casein and soy proteins, or peanuts.

6. The nutritional food or nutritional product of claim 1 further comprising at least one vitamin component and at least one mineral component.

7. The nutritional food or nutritional product of claim 1 wherein at least one prebiotic ingredient is a fructo-oligosaccharide, a galacto-oligosaccharide, a soy-oligosaccharide, a xylo-oligosaccharide, a isomalto-oligosaccharides, Jerusalem artichoke flour, rolled oats, banana fiber, a pectin and pectic polysaccharide, a mannan, a pentosan, a beta-glucan, a rabinan or a galactan.

8. A nutritional food or nutritional product for maintaining or enhancing gastrointestinal health, comprising at least one carbohydrate ingredient, at least one fat ingredient, at least one protein ingredient, at least one vitamin component, at least one mineral component, at least one prebiotic ingredient, and at least one probiotic bacteria, wherein said probiotic bacteria have a propensity to hydrolyze nitrogenous waste products.

9. A method of restoring and maintaining gastrointestinal health comprising administering to a subject at least one food or nutritional product comprising an effective amount of probiotic bacteria and an effective amount of a prebiotic.

10. A nutriceutical composition to alleviate the symptoms of uremia comprising a composition of a probiotic, a prebiotic, and an ammoniaphilic urea degrading microorganism with pH stability and urea degrading activity.

11. A yogurt or yogurt based product comprising at least one probiotic bacteria, at least one carbohydrate ingredient and at least one protein.
